SEQUENCE LISTING (4 sequences)

SEQ ID NO: 1 - DNA, 31 bp, Artificial
  Synthetic
catgccatggatcaacaaagcgccttctatg

SEQ ID NO: 2 - DNA, 37 bp, Artificial
  Synthetic
ataagaatgcggccgcaggcttgttgttgtcttcctc

SEQ ID NO: 3 - DNA, 695 bp, Artificial
  Synthetic
atcaacaaagcgccttctatgaaatcttgaacatgcctaacttaaacgaagagcaacgcaatggtttcattcaaagtcttaaagacgatccaagccaaagcactaacgttttaggtgaagctaaaaaattaaacgaatctcaagcaccgaaagctgacaacaatttcaacaaagaacaacaaaatgctttctatgaaatcttgaacatgcctaacttgaacgaagaacaacgcaatggtttcatccaaagcttaaaagatgacccaagtcaaagtgctaaccttttagcagaagctaaaaagttaaatgaatctcaagcaccgaaagctgataacaaattcaacaaagaacaacaaaatgctttctatgaaatcttacatttacctaacttaaatgaagaacaacgcaatggtttcatccaaagcttaaaagatgacccaagccaaagcgctaaccttttagcagaagctaaaaagctaaatgatgcacaagcaccaaaagctgacaacaaattcaacaaagaacaacaaaatgctttctatgaaattttacatttacctaacttaactgaagaacaacgtaacggcttcatccaaagccttaaagacgatccttcagtgagcaaagaaattttagcagaagctaaaaagctaaacgatgctcaagcaccaaaagaggaagacaacaacaagcct

SEQ ID NO: 4 - protein, 235 aa, Artificial
  Synthetic
MQQSAFYEILNMPNLNEEQRNGFIQSLKDDPSQSTNVLGEAKKLNESQAPKADNNFNKEQQNAFYEILNMPNLNEEQRNGFIQSLKDDPSQSANLLAEAKKLNESQAPKADNKFNKEQQNAFYEILHLPNLNEEQRNGFIQSLKDDPSQSANLLAEAKKLNDAQAPKADNKFNKEQQNAFYEILHLPNLTEEQRNGFIQSLKDDPSVSKEILAEAKKLNDAQAPKEEDNNKPAAA

(19) United States
(12) Patent Application Publication
(10) Pub. No.: US 2009/0317324 A1
(43) Pub. Date: Dec. 24, 2009

(54) FILAMENTOUS BACTERIOPHAGE DISPLAYING PROTEIN AS A BINDER OF ANTIBODIES AND IMMUNOCOMPLEXES FOR DELIVERY TO THE BRAIN

(75) Inventors: Beka Solomon, Herzlia Pituach (IL); Rachel Cohen Kupiec, Tel Aviv (IL)

Correspondence Address: BROWDY AND NEIMARK, P.L.L.C.;624 NINTH STREET, NW, SUITE 300, WASHINGTON, DC, 20001-5303, US

(73) Assignee: RAMOT at Tel Aviv University, Tel Aviv (IL)

(21) Appl. No.: 12/279,689

(22) PCT Filed: Feb. 15, 2007
(86) PCT No.: PCT/US07/62238

§ 371 Date: Aug. 26, 2009

Related U.S. Application Data
(60) Provisional application No. 60/773,320, filed on Feb. 15, 2006.

(51) Int. Cl. A61K 51/10; C12N 7/00; A61K 39/395; A61P 25/00

(52) U.S. Cl. 424/1.49; 435/235.1; 424/130.1

(57) ABSTRACT

The present invention relates to a phage display vehicle composed of a filamentous bacteriophage displaying on its surface, as a non-filamentous bacteriophage molecule, protein A or a fragment or variant thereof capable of binding the Fc portion of antibodies, and an antibody or an antigen-antibody immunocomplex bound to protein A or a fragment or variant thereof by its Fc portion. The phage display vehicle is formulated into a pharmaceutical composition and can be used to treat/inhibit or to diagnose a brain disease, disorder or condition.

### BACKGROUND OF THE INVENTION

1. Field of the Invention

The invention relates to a filamentous bacteriophage display vehicle for delivery of antibodies and immunocomplexes to the brain and its use in diagnostics and therapeutics.

2. Description of the Related Art

### Phage Display:

Combinatorial phage display peptide libraries provide an effective means to study protein:protein interactions. This technology relies on the production of very large collections of random peptides associated with their corresponding genetic blueprints (Scott et al, 1990; Dower, 1992; Lane et al, 1993; Cortese et al, 1994; Cortese et al, 1995; Cortese et al, 1996). Presentation of the random peptides is often accomplished by constructing chimeric proteins expressed on the outer surface of filamentous bacteriophages such as M13, fd and f1. This presentation makes the repertoires amenable to binding assays and specialized screening schemes (referred to as biopanning (Parmley et al, 1988)) leading to the affinity isolation and identification of peptides with desired binding properties. In this way peptides that bind to receptors (Koivunen et al, 1995; Wrighton et al, 1996; Sparks et al, 1994; Rasqualini et al, 1996), enzymes (Matthews et al, 1993; Schmitz et al, 1996) or antibodies (Scott et al, 1990; Cwirla et al, 1990; Felici et al, 1991; Luzzago et al, 1993; Hoess et al, 1993; Bonnycastle et al, 1996) have been efficiently selected.

Filamentous bacteriophages are nonlytic, male specific bacteriophages that infect Escherichia coli cells carrying an F-episome (for review, see Model et al, 1988). Filamentous phage particles appear as thin tubular structures 900 nm long and 10 nm thick containing a circular single stranded DNA genome (the +strand). The life cycle of the phage entails binding of the phage to the F-pilus of the bacterium followed by entry of the single stranded DNA genome into the host. The circular single stranded DNA is recognized by the host replication machinery and the synthesis of the complementary second DNA strand is initiated at the phage ori(−) structure. The double stranded DNA replicating form is the template for the synthesis of single-stranded DNA circular phage genomes, initiating at the ori(+) structure. These are ultimately packaged into virions and the phage particles are extruded from the bacterium without causing lysis or apparent damage to the host.

Peptide display systems have exploited two structural proteins of the phage; pIII (P3) protein and pVIII protein. The pIII protein exists in 5 copies per phage and is found exclusively at one tip of the virion (Goldsmith et al, 1977). The N-terminal domain of the pIII protein forms a knob-like structure that is required for the infectivity process (Gray et al, 1981). It enables the adsorption of the phage to the tip of the F-pilus and subsequently the penetration and translocation of the single stranded phage DNA into the bacterial host cell (Holliger et al, 1997). The pIII protein can tolerate extensive modifications and thus has been used to express peptides at its N-terminus. The foreign peptides have been up to 65 amino acid residues long (Bluthner et al, 1996; Kay et al, 1993) and in some instances even as large as full-length proteins (McCafferty et al, 1990; McCafferty et al, 1992) without markedly affecting pIII function.

The cylindrical protein envelope surrounding the single stranded phage DNA is composed of 2700 copies of the major coat protein, pVIII, an α-helical subunit which consists of 50 amino acid residues. The pVIII proteins themselves are arranged in a helical pattern, with the α-helix of the protein oriented at a shallow angle to the long axis of the virion (Marvin et al, 1994). The primary structure of this protein contains three separate domains: (1) the N-terminal part, enriched with acidic amino acids and exposed to the outside environment; (2) a central hydrophobic domain responsible for: (i) subunit:subunit interactions in the phage particle and (ii) transmembrane functions in the host cell; and (3) the third domain containing basic amino acids, clustered at the C-terminus, which is buried in the interior of the phage and is associated with the phage-DNA. pVIII is synthesized as a precoat protein containing a 23 amino acid leader-peptide, which is cleaved upon translocation across the inner membrane of the bacterium to yield the mature 50-residue transmembrane protein (Sugimoto et al, 1977). Use of pVIII as a display scaffold is hindered by the fact that it can tolerate the addition of peptides no longer than 6 residues at its N-terminus (Greenwood et al, 1991; Iannolo et al, 1995). Larger inserts interfere with phage assembly. Introduction of larger peptides, however, is possible in systems where mosaic phages are produced by in vivo mixing the recombinant, peptide-containing, pVIII proteins with wild type pVIII (Felici et al, 1991; Greenwood et al, 1991; Willis et al, 1993). This enables the incorporation of the chimeric pVIII proteins at low density (tens to hundreds of copies per particle) on the phage surface interspersed with wild type coat proteins during the assembly of phage particles. Two systems have been used that enable the generation of mosaic phages; the “type 8+8” and “type 88” systems as designated by Smith (Smith, 1993).

The “type 8+8” system is based on having the two pVIII genes situated separately in two different genetic units (Felici et al, 1991; Greenwood et al, 1991; Willis et al, 1993). The recombinant pVIII gene is located on a phagemid, a plasmid that contains, in addition to its own origin of replication, the phage origins of replication and packaging signal. The wild type pVIII protein is supplied by superinfecting phagemid-harboring bacteria with a helper phage. In addition, the helper phage provides the phage replication and assembly machinery that package both the phagemid and the helper genomes into virions. Therefore, two types of particles are secreted by such bacteria, helper and phagemid, both of which incorporate a mixture of recombinant and wild type pVIII proteins.

The “type 88” system benefits by containing the two pVIII genes in one and the same infectious phage genome. Thus, this obviates the need for a helper phage and superinfection. Furthermore, only one type of mosaic phage is produced.

The phage genome encodes 10 proteins (pI through pX) all of which are essential for production of infectious progeny (Felici et al, 1991). The genes for the proteins are organized in two tightly packed transcriptional units separated by two non-coding regions (Van Wezenbeek et al, 1980). One non-coding region, called the “intergenic region” (defined as situated between the pIV and pII genes) contains the (+) and the (−) origins of DNA replication and the packaging signal of the phage, enabling the initiation of capsid formation. Parts of this intergenic region are dispensable (Kim et al, 1981; Dotto et al, 1984). Moreover, this region has been found to be able to tolerate the insertion of foreign DNAs at several sites (Messing, 1983; Moses et al, 1980; Zacher et al, 1980). The second non-coding region of the phage is located between the pVIII and pIII genes, and has also been used to incorporate foreign recombinant genes as was illustrated by Pluckthun (Krebber et al, 1995).

Immunization with Phage Display:

Small synthetic peptides, consisting of epitopes, are generally poor antigens requiring the chemical synthesis of a peptide and need to be coupled to a large carrier, but even then they may induce a low affinity immune response. An immunization procedure for raising anti-AβP antibodies, using as antigen the filamentous phages displaying only EFRH peptide, was developed in the laboratory of the present inventors (Frenkel et al., 2000 and 2001). Filamentous bacteriophages have been used extensively in recent years for the ‘display’ on their surface of large repertoires of peptides generated by cloning random oligonucleotides at the 5′ end of the genes coding for the phage coat protein (Scott and Smith, 1990; Scott, 1992). As recently reported, filamentous bacteriophages are excellent vehicles for the expression and presentation of foreign peptides in a variety of biologicals (Greenwood et al., 1993; Medynski, 1994). Administration of filamentous phages induces a strong immunological response to the phage effects systems (Willis et al., 1993; Meola et al., 1995). Phage coat proteins pII and pVIII discussed above are proteins that have been often used for phage display.

Due to its linear structure, filamentous phage has high permeability to different kinds of membranes (Scott et al., 1990) and following the olfactory tract, it reaches the hippocampus area via the limbic system to target affected sites. The treatment of filamentous phage with chloroform changes the linear structure to a circular one, which prevents delivery of phage to the brain.

### Antibody Engineering:

Antibody engineering methods were applied to minimize the size of mAbs (135-900 kDa) while maintaining their biological activity (Winter et al., 1994). These technologies and the application of the PCR technology to create large antibody gene repertoires make antibody phage display a versatile tool for isolation and characterization of single chain Fv (scFv) antibodies (Hoogenboom et al., 1998). The scFvs can be displayed on the surface of the phage for further manipulation or may be released as a soluble scfv (˜25 kd) fragment. The laboratory of the present inventors have engineered an scFv which exhibits anti-aggregating properties similar to the parental IgM molecule (Frenkel et al., 2000a). For scFv construction, the antibody genes from the anti-AβP IgM 508 hybridoma were cloned. The secreted antibody showed specific activity toward the AβP molecule in preventing its toxic effects on cultured PC 12 cells. Site-directed single-chain Fv antibodies are the first step towards targeting therapeutic antibodies into the brain via intracellular or extracellular approaches.

### Protein A:

Protein A of Staphylococcus aureus is a cell wall constituent characterized by its affinity to the Fc portion of immunoglobulins, especially the IgG class (Goding, 1978). It binds IgG antibodies of humans, mice, pigs, guinea pigs and rabbits. In mice, protein A binds IgG2a and IgG2b antibodies in high affinity, but binds IgG1 and IgG3 antibodies less well (Goudswaard et al., 1978). Protein A is a 42 kDa protein that has four repetitive domains rich in aspartic and glutamic acids but devoid of cysteines. The IgG binding domain (domain B) consists of three anti-parallel alpha-helicies, the third of which is disrupted when the protein is complexed with Fc (Graille et al., 2000).

### Plaque-Forming Diseases:

Plaque forming diseases are characterized by the presence of amyloid plaque deposits in the brain as well as neuronal degeneration. Amyloid deposits are formed by peptide aggregated into an insoluble mass. The nature of the peptide varies in different diseases but in most cases, the aggregate has a beta-pleated sheet structure and stains with Congo Red dye. In addition to Alzheimer's disease (AD), which includes early onset Alzheimer's disease, late onset Alzheimer's disease, and presymptomatic Alzheimer's disease, other diseases characterized by amyloid deposits are, for example, SAA amyloidosis, hereditary Icelandic syndrome, multiple myeloma, and prion diseases. The most common prion diseases in animals are scrapie of sheep and goats and bovine spongiform encephalopathy (BSE) of cattle (Wilesmith and Wells, 1991). Four prion diseases have been identified in humans: (i) kuru, (ii) Creutzfeldt-Jakob Disease (CJD), (iii) Gerstmann-Streussler-Sheinker Disease (GSS), and (iv) fatal familial insomnia (FFI) (Gajdusek, 1977; and Tritschler et al. 1992).

Prion diseases involve conversion of the normal cellular prion protein (PrPC) into the corresponding scrapie isoform (PrPSc). Spectroscopic measurements demonstrate that the conversion of PrPC into the scrapie isoform (PrPSc) involves a major conformational transition, implying that prion diseases, like other amyloidogenic diseases, are disorders of protein conformation. The transition from PrPC to PrPSc is accompanied by a decrease in 1-helical secondary structure (from 42% to 30%) and a remarkable increase in β-sheet content (from 3% to 43%) (Caughey et al, 1991; and Pan et al, 1993). This rearrangement is associated with abnormal physiochemical properties, including insolubility in non-denaturing detergents and partial resistance to proteolysis. Previous studies have shown that a synthetic peptide homologous with residues 106-126 of human PrP (PrP106-126) exhibits some of the pathogenic and physicochemical properties of PrPSc (Selvaggini et al, 1993; Tagliavini et al, 1993; and Forloni et al, 1993). The peptide shows a remarkable conformational polymorphism, acquiring different secondary structures in various environments (De Gioia et al, 1994). It tends to adopt a β-sheet conformation in buffered solutions, and aggregates into amyloid fibrils that are partly resistant to digestion with protease. X-ray crystallographic studies of a complex of antibody 3F4 and its peptide epitope (PrP 104-113) provided a structural view of this flexible region that is thought to be a component of the conformational rearrangement essential to the development of prion disease (Kanyo et al, 1999).

Alzheimer's disease (AD) is a progressive disease resulting in senile dementia. Broadly speaking, the disease falls into two categories: late onset, which occurs in old age (typically above 65 years) and early onset, which develops well before the senile period, e.g., between 35 and 60 years. In both types of the disease, the pathology is similar, but the abnormalities tend to be more severe and widespread in cases beginning at an earlier age. The disease is characterized by two types of lesions in the brain, senile plaques and neurofibrillary tangles. Senile plaques are areas of disorganized neutrophils up to 150 mm across with extracellular amyloid deposits at the center, visible by microscopic analysis of sections of brain tissue. Neurofibrillary tangles are intracellular deposits of tau protein consisting of two filaments twisted about each other in pairs.

The principal constituent of the senile plaques is a peptide termed amyloid beta (Aβ) or beta-amyloid peptide (βAP or βA). The amyloid beta peptide is an internal fragment of 39-43 amino acids of a precursor protein termed amyloid precursor protein (APP). Several mutations within the APP protein have been correlated with the presence of Alzheimer's disease (Goate et al, (1991), valine717 to isoleucine; Chartier Harlan et al, (1991), valine717 to glycine; Murrell et al, (1991), valine717 to phenylalanine; Mullan et al, (1992), a double mutation, changing lysine595-methionine596 to asparagine595-leucine596).

Such mutations are thought to cause Alzheimer's disease by increased or altered processing of APP to beta-amyloid, particularly processing of APP to increased amounts of the long form of beta-amyloid (i.e., Aβ1-42 and Aβ1-43). Mutations in other genes, such as the presenilin genes, PS1 and PS2, are thought indirectly to affect processing of APP to generate increased amounts of long form beta-amyloid (see Hardy, TINS 20, 154, 1997). These observations indicate that beta-amyloid, and particularly its long form, is a causative element in Alzheimer's disease.

Publications on amyloid fibers indicate that cylindrical β-sheets are the only structures consistent with some of the x-ray and electron microscope data, and fibers of Alzheimer Aβ fragments and variants are probably made of either two or three concentric cylindrical β-sheets (Perutz et al., 2002). The complete Aβ peptide contains 42 residues, just the right number to nucleate a cylindrical shell; this finding and the many possible strong electrostatic interactions in β-sheets made of the Aβ peptide in the absence of prolines account for the propensity of the Aβ peptide to form the extracellular amyloid plaques found in Alzheimer patients. If this interpretation is correct, amyloid consists of narrow tubes (nanotubes) with a central water-filled cavity. Reversibility of amyloid plaque growth in-vitro suggests steady-state equilibrium between βA in plaques and in solution (Maggio and Mantyh, 1996). The dependence of βA polymerization on peptide-peptide interactions to form a β-pleated sheet fibril, and the stimulatory influence of other proteins on the reaction, suggest that amyloid formation may be subject to modulation. Many attempts have been made to find substances able to interfere with amyloid formation. Among the most investigated compounds are antibodies, peptide composed of beta-breaker amino acids like proline, addition of charged groups to the recognition motif and the use of N-methylated amino-acid as building blocks (reviewed by Gazit, 2002).

Methods for the prevention or treatment of diseases characterized by amyloid aggregation in a patient have been proposed which involve causing antibodies against a peptide component of an amyloid deposit to come into contact with aggregated or soluble amyloid. See WO99/27944 of Schenk and U.S. Pat. No. 5,688,651 of Solomon, the entire contents of each being herein incorporated by reference. The antibodies may be caused to come into contact with the soluble or aggregated amyloid by either active or passive vaccination. In active vaccination, a peptide, which may be an entire amyloid peptide or a portion thereof, is administered in order to raise antibodies in vivo, which antibodies will bind to the soluble and/or the aggregated amyloid. Passive vaccination involves administering antibodies specific to the amyloid peptide directly. These procedures are preferably used for the treatment of Alzheimer's disease by diminishing the amyloid plaque or slowing the rate of deposition of such plaque.

It has been reported that clinical trials of a vaccine to test such a process had been undertaken by Elan Corporation and Wyeth-Ayerst Laboratories. The compound being tested was AN-1792. This product has been reported to be a form of β-amyloid 42.

Citation of any document herein is not intended as an admission that such document is pertinent prior art, or considered material to the patentability of any claim of the present application. Any statement as to content or a date of any document is based on the information available to applicant at the time of filing and does not constitute an admission as to the correctness of such a statement.

### SUMMARY OF THE INVENTION

The present invention provides a phage display vehicle containing a filamentous bacteriophage displaying on its surface protein A, or a fragment or variant thereof capable of binding the Fc portion of antibodies, as a non-native filamentous bacteriophage molecule, and an antibody or an antigen-antibody immunocomplex bound to the protein A or fragment or variant thereof by its Fc portion.

The present invention also provides a pharmaceutical composition containing the phage display vehicle of the present invention for use as a therapeutic or diagnostic.

Further provided by the present invention are methods for treating/inhibiting or for diagnosing a brain disease, disorder or condition by intranasally administering the phage display vehicle of the present invention to a subject in need thereof.

### BRIEF DESCRIPTION OF THE DRAWINGS

FIG. 1A shows the primers ProtA-fwd (SEQ ID NO:1) and ProtA-rev (SEQ ID NO:2), used for the PCR synthesis of a protein A variant. FIG. 1B shows the nucleotide sequence (SEQ ID NO:3) of the protein A variant. FIG. 1C shows the amino acid open reading frame of the protein A variant (SEQ ID NO:4).

FIG. 2 is a graph showing that phage-protein A binds antibodies of IgG1 type (196, 10D5, 6C6 and 2H3). It also binds IgG2a (3D6) and weakly to IgG2b (12B4).

FIG. 3 is a graph showing that phage-ProtA bound antibodies to Aβ1-16 did not detach from protein A once they bind to Aβ1-16.

FIG. 4 is a graph showing the titration of phage-protA1 binding to mAb196.

FIGS. 5A and 5B are graphs showing the titration of phage with two separate concentrations of antibody 2H3.

FIG. 6 is a graph showing the results of ELISA to check phage protein A disassociation from antibody 196 following PEG precipitation. Treatments were: 1, Phage-Protein A combined with 5 μg 196 and PEG precipitated; 2, The same as 1 without PEG precipitation; 3, Only antibody 196—PEG precipitated; 4, as in 3 but without PEG precipitation; 4, Phage—protein A only without antibody and without PEG precipitation.

FIGS. 7A and 7B are graphs showing the binding of phage-PrtA-anti-insulin to insulin (FIG. 7A) and the binding of phage-PrtA-insulin-anti-insulin immunocomplexes detected with goat anti-mouse antibodies (FIG. 7B).

FIG. 8 is a graph comparing levels of soluble and insoluble Aβ1-42 and insoluble Aβ1-40 in mice treated with the phage complexes and control untreated mice.

FIGS. 9A and 9B are graphs showing the levels of insoluble and soluble Aβ1-42 in PDAPP mice treated with the phage-196 complex compared to untreated control mice.

FIGS. 10A and 10B are graphs showing cytokine levels of IL1β and IL10 in complex treated mice and non-transgenic and transgenic control mice.

### DETAILED DESCRIPTION OF THE INVENTION

Filamentous bacteriophages have a linear structure which enables them to penetrate the brain when applied intranasally. As they can be engineered to present assorted proteins or peptides on their coat proteins, they can be used to deliver antibodies or molecules (in the form of an immunocomplex with their specific antibodies) into the brain of the mammalian subject. P3 is a structural protein that assembles one of the tips of the phage coat through which the phage invades the bacteria Escherichia coli.

One aspect of the present invention is directed to a phage display vehicle which includes a filamentous bacteriophage that displays on its surface, as a non-native filamentous bacteriophage molecule, protein A or a fragment or variant thereof capable of binding the Fc portion of antibodies and further includes an antibody or antigen-antibody immunocomplex bound to protein A or a fragment or variant thereof by its Fc portion. In a preferred embodiment, the filamentous bacteriophage is not also conjugated through a linker or directly to a drug and the antibody or the antigen-antibody immunocomplex is not also immobilized on a solid carrier. In another preferred embodiment, the antibody bound to protein A or a fragment or variant thereof displayed on the filamentous bacteriophage is specific for a molecule that is not presented as a target on a cell surface and the antibody is not immobilized on a solid carrier.

In the laboratory of the present inventors, filamentous phages M13, f1, and fd, which are well understood at both structural and genetic levels (Greenwood et al., 1991) were used. This laboratory first showed that filamentous bacteriophage exhibits penetration properties to the central nervous system while preserving both the inert properties of the vector and the ability to carry foreign molecules (Frenkel and Solomon, 2002).

Filamentous bacteriophages are a group of structurally related viruses which contain a circular single-stranded DNA genome. They do not kill their host during productive infection. The phages that infect Escherichia coli containing the F plasmids are collectively referred to as Ff bacteriophages. They do not infect mammalian cells.

The filamentous bacteriophages are flexible rods about 1 to 2 microns long and 6 nm in diameter, with a helical shell of protein subunits surrounding a DNA core. The two main coat proteins, protein pIII and the major coat protein pVIII, differ in the number of copies of the displayed protein. While pIII is presented in 4-5 copies, pVIII is found in ˜3000 copies. The approximately 50-residue major coat protein pVIII subunit is largely alpha-helical and the axis of the alpha-helix makes a small angle with the axis of the virion. The protein shell can be considered in three sections: the outer surface, occupied by the N-terminal region of the subunit, rich in acidic residues that interact with the surrounding solvent and give the virion a low isoelectric point; the interior of the shell, including a 19-residue stretch of a polar side-chains, where protein subunits interact mainly with each other; and the inner surface, occupied by the C-terminal region of the subunit, rich in basic residues that interact with the DNA core. The fact that virtually all protein side-chain interactions are between different subunits in the coat protein array, rather than within subunits, makes this a useful model system for studies of interactions between alpha-helical subunits in a macromolecular assembly. The unique structure of filamentous bacteriophage enables its penetration into the brain, although it has a mass of approximately 16.3MD and may contribute to its ability to interfere with βA fibrillization since the phage structure resemble an amyloid fibril itself.

The filamentous bacteriophage can be any filamentous bacteriophage such as M13, f1, or fd. Although M13 was used in the Example hereinbelow, any other filamentous bacteriophage is expected to behave and function in a similar manner as they have similar structure and as their genomes have greater than 95% genome identity.

In the phage display vehicle according to the present invention, protein A, or a fragment or variant thereof, is displayed on the surface of the filamentous bacteriophage. This phage display involves the expression of a cDNA clone of protein A or a fragment or variant thereof as a fusion protein with a phage coat protein. Thus, the filamentous bacteriophage genome is genetically modified to display a normative polypeptide or peptide on its surface. Filamentous bacteriophages that display foreign proteins or peptides as a fusion with a phage coat protein are well known to those in the art. A variety of phages and coat proteins may be used, including, but not limited to: M13 protein III, M13 protein VIII, M13 protein VI, M13 protein VI, M13 protein IX, and fd minor coat protein pIII (Saggio et al., 1995; Uppala and Koivunen, 2000). A large array of vectors are available (see Kay et al., 1996; Berdichevsky et al., 1999; and Benhar, 2001). In a preferred embodiment, protein A or a fragment or variant thereof is displayed by its fusion to the minor coat protein (protein III) of a filamentous phage. Methods for inserting foreign coding sequences into a phage gene are well known (see e.g., Sambrook et al., 1989; and Brent et al., 2003).

Protein A of Staphylococcus aureus is a well known and well used protein in the art for binding the Fc portion of antibodies. Fragments of protein containing the binding domain(s) are also well recognized in the art. There is also a wealth of knowledge concerning variants of protein A with improved binding (or differential binding to Fc from different Ig classes and subclasses) to the Fc portion of antibodies (immunoglobulins). Preferably, the antibody bound to the protein A or fragment or variant thereof is of the IgG class. A most preferred embodiment of a protein A variant is the variant having the amino acid sequence of SEQ ID NO:4.

The protein A or a fragment or variant thereof capable of binding the Fc portion of antibodies is displayed on the surface of a filamentous bacteriophage to bind an antibody or an antigen-antibody immunocomplex for delivery to the brain.

The present invention provides a method for inhibiting or treating a brain disease, disorder or condition by introducing the phage delivery vehicle to the brain. In addition, the present method further provides a method for diagnosing a brain disease, disorder or condition by introducing a phage display vehicle to the brain which can be detected, i.e., labeled. The present methods involve introducing/administering to a subject in need thereof an effective amount of the phage display vehicle of the present invention.

For purposes of this specification and the accompanying claims, the terms “patient”, “subject” and “recipient” are used interchangeably. They include humans and other mammals which are the object of either prophylactic or therapeutic treatment, or of diagnosis.

For purposes of this specification and the accompanying claims, the terms “beta amyloid peptide” is synonymous with “β-amyloid peptide”, “amyloid β peptide” “βAP”, “βA”, and “Aβ”. All of these terms refer to a plaque forming peptide derived from amyloid precursor protein. In a preferred embodiment, the antibody bound to protein A (or a fragment or variant thereof) displayed on the phage surface is specific for (binds specifically to) an amyloid β peptide. The amyloid β peptide is preferably Aβ1-42 or Aβ1-40.

As used herein, “PrP protein”, “PrP”, “prion”, refer to polypeptides which are capable under appropriate conditions, of inducing the formation of aggregates responsible for plaque forming diseases. For example, normal cellular prion protein (PrPC) is converted under such conditions into the corresponding scrapie isoform (PrPSc) which is responsible for plaque forming diseases such as, but not limited to, bovine spongiform encephalopathy (BSE), or mad cow disease, feline spongiform encephalopathy of cats, kuru, Creutzfeldt-Jakob Disease (CJD), Gerstmann-Straussler-Scheinker disease (GSS), and fatal familial insomnia (FFI). In another preferred embodiment, the antibody is specific for the pathogenic PrPSc isoform.

The term “treating” is intended to mean substantially inhibiting, slowing or reversing the progression of a disease, disorder or condition, substantially ameliorating clinical symptoms of a disease, disorder or condition or substantially preventing the appearance of clinical symptoms of a disease, disorder or condition.

The phage delivery vehicle and the methods of the present invention are directed to a brain disease, disorder or condition. Preferably, the brain disease, disorder or condition is a “plaque-forming disease”.

The term “plaque forming disease” refers to diseases characterized by formation of plaques by an aggregating protein (plaque forming peptide), such as, but not limited to, beta-amyloid, serum amyloid A, cystatin C, IgG kappa light chain or prion protein, in diseases such as, but not limited to, early onset Alzheimer's disease (AD), late onset Alzheimer's disease, presymptomatic Alzheimer's disease, SAA amyloidosis, hereditary Icelandic syndrome, senility, multiple myeloma, and to prion diseases that are known to affect humans, such as for example, kuru, Creutzfeldt-Jakob disease (CJD), Gerstmann-Straussler-Scheinker disease (GSS), and fatal familial insomnia (FFI) and animals, such as, for example, scrapie and bovine spongiform encephalitis (BSE).

Because most of the amyloid plaques (also known as amyloid deposits) associated with the plaque-forming diseases described hereinabove are located within the brain, any proposed treatment modality must demonstrate an ability to cross the blood brain barrier (BBB) as well as an ability to dissolve amyloid plaques Normally, the average size of molecules capable of penetrating the BBB is approximately 2 kDa.

An increasing body of evidence shows that olfactory deficits and degenerative changes in the central olfactory pathways are affected early in the clinical course of AD. Moreover, the anatomic patterns involved in AD suggest that the olfactory pathway may be the initial stage in the development of AD.

Olfactory receptor neurons are bipolar cells that reside in the epithelial lining of the nasal cavity. Their axons traverse the cribriform plate and project to the first synapse of the olfactory pathway in the olfactory bulb of the brain. The axons of olfactory neurons from the nasal epithelium form bundles of 1000 amyelinic fibers. This configuration makes them a highway by which viruses or other transported substances may gain access to the CNS across the blood brain barrier (BBB).

As previously shown, intranasal administration (Mathison et al, 1998; Chou et al, 1997; Draghia et al, 1995) enables the direct entry of viruses and macromolecules into the cerebrospinal fluid (CSF) or CNS.

Use of olfactory receptor neurons as a point of delivery for an adenovirus vector to the brain is reported in the literature. This method reportedly causes expression of a reporter gene in the brain for 12 days without apparent toxicity (Draghia et al, 1995).

The direct brain delivery of antibodies or immunocomplexes overcomes crossing the BBB by using olfactory neurons as transporters to the brain. In the olfactory epithelium, the dendrites of the primary olfactory neurons are in contact with the nasal lumen, and via the axons, these neurons are also connected to the olfactory bulbs of the brain. Phages that come into contact with the olfactory epithelium can be taken up in the primary olfactory neurons and be transported to the olfactory bulbs, and even further into other areas of the brain.

Also included in brain diseases, disorders or conditions treated/inhibited or diagnosed according to the present invention are brain tumors and brain inflammatory diseases, disorders or conditions. Brain inflammation causes inhibition of neurogenesis both in the basal continuous formation of new neurons in intact hippocampal formation and in increased neurogenesis in response to a brain insult. Impairment of neurogenesis depends on the degree of microglia activation, irrespective of whether there is damage or not in the surrounding tissue. Brain inflammation probably plays an important role in the pathogenesis of other chronic neurodegenerative disorders besides AD, which involves AβP pathological effects, like Parkinson's disease, Lewy Body Dementia, AIDS Dementia Complex, traumatic brain injury, glaucoma, etc.

Traumatic brain injury (TBI) includes β-amyloid deposition and the early onset of dementia. Despite such descriptions, little is known about the mechanisms through which these changes occur. One source proposed for the generation of β-amyloid peptide in TBI is abnormal proteolytic cleavage of the β-amyloid precursor protein (APP) that has been shown to accumulate at sites of impaired axoplasmic transport within traumatically injured axons. In fact, βA immunoreactivity has recently been found with swollen axons in a pig model of TBI (Smith et al., 1999), suggesting the accumulation of APP in traumatically injured axons may be a source for β-amyloid peptide formation in TBI.

Non-limiting examples of neurodegenerative diseases and disorders include Alzheimer's disease (AD), Parkinson's disease, Lewy Body Dementia, AIDS Dementia Complex, stroke, and closed head injuries and traumatic brain injury, such as gunshot wounds, hemorrhagic stroke, ischemic stroke, cerebral ischemia, damages caused by nerve damaging agents such as toxins, poisons, chemical (biowarfare) agents or damages caused by surgery such as tumor excision.

In a preferred embodiment, the antibody bound to the phage displayed protein A (or fragment or variant thereof) is a monoclonal antibody specific for a target molecule associated with a brain disease, disorder or condition, such as amyloid D peptide and PrPSc. The antibody can also be an antibody against inflammatory cytokines in the brain, such as TNFα, IL6 etc., to inhibit/treat brain inflammation as a result of stroke, brain injury or other neurodegenerative disease or disorder. In the case of the method for diagnosing a brain tumor as the brain disease, disorder or condition, the antibody is specific for a target molecule characteristic of the brain tumor and whose presence is diagnostic for the particular brain tumor.

Alternatively, the phage delivery vehicle displaying protein A (or a fragment or variant thereof) bound to an antigen-antibody immunocomplex by the Fc portion of the antibody can be used to deliver the immunocomplex into the brain. The antigen bound by the antibody in the immunocomplex is a molecule that can treat a brain disease disorder or condition. Non-limiting examples of the antigen bound by the antibody include insulin, erythropoietin (EPO) which can decrease neuronal injury and cell death, interferon and other anti-inflammatory cytokines such as IL10 and neuronal protective molecules which do not pass through the blood brain barrier.

The method for diagnosing a brain disease, disorder or condition according to the present invention involves intranasally administering the phage display vehicle of the present invention to a subject in need thereof. When the phage display vehicle with its antibody component is bound to a target molecule associated with a brain disease, disorder or condition, the presence of the brain disease, disorder or condition is then detected. The antibody bound to the protein A (or fragment or variant thereof capable of binding to the Fc portion of an antibody) displayed on the surface of the filamentous bacteriophage can be detectably labeled.

Antibodies that are labeled are preferably radiolabeled for use in therapy against brain tumors or in diagnostic radioimaging. Non-limiting examples of radionuclides for labeling include ¹¹¹In, ¹²⁵I, ¹³¹I and ⁹⁹mTc. The radiolabeled antibodies, which are preferably radioiodinated antibodies, are prepared by standard methods for radiolabeling peptides and proteins, and are then bound to protein A (or a fragment or variant thereof) displayed on the surface of filamentous bacteriophage.

Other suitable detectable labels include contrast agents such as gadolinium (Gd), which is a preferred reagent for enhanced dynamic MRI, and organic or inorganic compounds of a heavy element (e.g., Pt, Au, and Tl).

A pharmaceutical preparation according to the present invention includes, as an active ingredient, a phage display vehicle of the present invention. The pharmaceutical preparation can also be a mixture of phage display vehicles from different filamentous bacteriophages.

The preparation according to the present invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

The term “active ingredient” refers to the preparation accountable for the biological effect. In the context of a pharmaceutical composition for use in a diagnostic application, the term “active ingredient” is intended to be the antibody targeting a molecule associated with a brain disease, disorder or condition.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound.

The term “excipient” refers to an inert substance added to a pharmaceutical composition to further facilitate administration of an active ingredient. Non-limiting examples, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which can be used pharmaceutically.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. A nasal spray, which does not require a pressurized pack or nebulizer as in an inhalation spray, can alternatively used for intranasal administration. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

Pharmaceutical compositions suitable for use in the context of the present invention include compositions wherein the active ingredients are contained in an amount effective to achieve the intended purpose. More specifically, a therapeutically effective amount means an amount of active ingredients effective to prevent, alleviate or ameliorate symptoms of a disease, disorder or condition, or prolong the survival of the subject being treated.

Determination of a therapeutically or diagnostically effective amount is well within the capability of those skilled in the art, especially in light of the detailed disclosure provided herein.

Dosage amount and interval may be adjusted individually to provide brain levels of the phage display vehicle which are sufficient to treat or diagnose a particular brain disease, disorder, or condition (minimal effective concentration, MEC). The MEC will vary for each preparation, but can be estimated from in vitro data. Dosages necessary to achieve the MEC will depend on individual characteristics.

Dosage intervals can also be determined using the MEC value. Preparations should be administered using a regimen, which maintains brain levels above the MEC for 10-90% of the time, preferable between 30-90% and most preferably 50-90%.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated or diagnosed, the severity of the affliction, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as if further detailed above.

Having now generally described the invention, the same will be more readily understood through reference to the following example which is provided by way of illustration and is not intended to be limiting of the present invention.

### Example

Filamentous phages that display a variant of protein A on P3 were constructed in this study. Previous works have used this basic idea, however, in different ways: Li et al. (1998) used phage displaying the B domain of protein A fused to a scFv molecule. Djojonegoro et al. (1994) used only domain B of protein A displayed on M13 phage, and Sampath et al. (1997) used the B domain to demonstrate that filamentous phages can be used as cloning tools. All these previous studies used phage display techniques to enable easy selection of improved forms of protein A displayed on phage by binding the protein A IgG binding domain to human IgGs. The experiments presented below in this study show that phages that display the protein A variant bind mouse antibodies, mainly of the IgG1 type and their ligands. These complexes were administered by intranasal application to hAPP tg mice. They entered the mice brains and exerted effects that are discussed below.

### Materials and Methods

### Synthesis of Protein A Variant

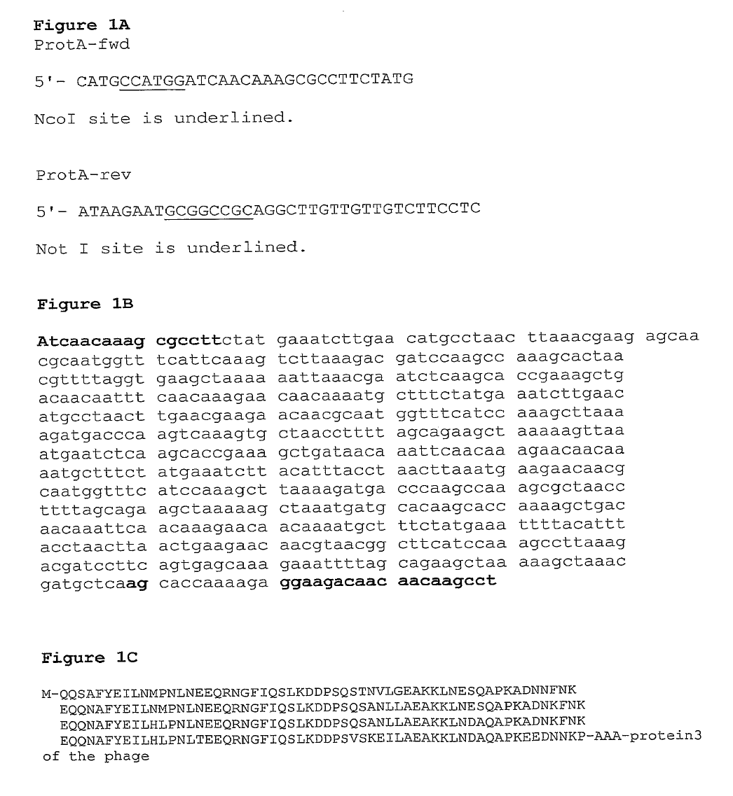

The portion of protein A that was used contains the four repetitive domains, including domain B. Two primers were synthesized, the forward primer with an ATG codon and an NcoI restriction site, and a reverse primer without a stop codon and with a NotI restriction site (FIG. 1A). PCR was performed using the following protocol: 2 μg genomic DNA of Staphylococcus aureus was combined with 20 μM of each primer in 1× buffer of Qiagen Taq polymeras. dNTPs (0.2 mM), and 2.5 units of the polymerase were added to 100 μl reaction volume. PCR conditions were: 3 min initial denaturation at 94° C. followed by 30 cycles of 30 seconds denaturation at 94° C., 1 min annealing at 55° C. and 1 min polymerization at 72° C. The PCR product was digested with NcoI and NotI, gel purified and cloned in the vector pCANTAB 5E that was previously linearized with NcoI and NotI. The cloning of this portion of the protein A gene in this vector generated a translational fusion of protein A with gene 3 of the filamentous phage (M13). Using helper phage, phages that display the protein A-P3 fusion, designated phage protA were produced.

### ELISA

Binding of phage protein A to different IgG antibodies was measured by ELISA: 10¹¹ phages displaying protein A were mixed with 10 μg (15 nmols) of antibody in 0.1 M sodium phosphate, pH 8.5. The mixture was gently shaken at 37° C. for 30 minutes, and 50 μl aliquots were then added in tetraplicates to ELISA wells previously coated with rabbit anti-phage antibody and blocked with 3% milk. Goat anti-mouse-HRP conjugated antibody was used as a secondary antibody. HRP reaction was carried out with OPD and peroxide. Results are depicted as OD at 492 nm.

Stability of phage-protein A-antibody complexes following antigen binding was checked by the following procedure: 10¹¹ phages displaying protein A were mixed with 10 μg (15 nmols) of antibody in 0.1 M sodium phosphate pH 8.5. The mixture was gently shaken at 37° C. for 30 minutes and the antigen, (0.32 nmols of biotinylated Abeta 1-16) at molar ratio of 50:1 in favor of the antibody, was then added to the mixture of phage-protein A and antibodies for an additional 30 minutes. Aliquots (50 μl) were added in tetraplicates to ELISA wells previously coated with rabbit anti-phage antibody and blocked with 3% milk. Avidin-HRP conjugate was used to detect complexes that bound the ELISA plate. Only phage-antibody complexes that were still bound to the antigen would react with the Avidin-HRP conjugate. The serum used in these experiments at a dilution of 1:1000 was drawn from a mouse with titer against ERFH epitope.

To titrate the number of phages required to bind different amounts of antibodies, antibody 196, which demonstrated the best binding capacity to protein A, was used to coat ELISA plates in 3 different concentrations (50, 100 and 250 ng/well), followed by blocking the wells with 3% milk. Phage-protein A in different concentrations (10⁹/ml-10¹²/ml) were added and the bound phages were detected by rabbit anti-phage antibodies and goat anti-rabbit-HRP antibodies.

The binding of antibody-antigen complexes to phage-protein A was titrated as follows: 10⁹/ml to 10¹²/ml phages were combined with two separate concentrations of antibody 2H3, 10 ng or 50 ng, and 1:2 molar ratios of antibody:antigen (two antigen molecules for every antibody molecule). The mixtures were gently shaken at 37° C. for 30 minutes and then added to ELISA wells, previously coated with rabbit anti-phage antibodies and coated with 3% milk in PBS. Detection of the bound phages was done with Avidin-HRP conjugate, which can only bind the biotinylated antigens.

Whether or not PEG precipitation of phage-protein A antibody complexes cause their dissociation was also checked: ELISA wells were coated with 20 μg/ml avidin overnight at 40° C. and then with 1 μg/ml biotinylated Aβ 1-16 peptide for 30 minutes at room temperature. The wells were blocked with 3% milk in PBS. 10¹² phages were combined with 1 μg or with 5 μg antibody 196 in 1 ml 0.1 M Na₂HPO₄ and gently shaken for 1 hr at 37° C. Half the mixture was precipitated using PEG-NaCl, followed by resuspension in 500 μl 0.1 M Na₂HPO₄ as before, and the other half was used without precipitation. Both mixtures were applied to the previously described ELISA plate (50 μl mixture/well) for 1 hr at 37° C. Detection of the bound phages was carried out with mouse anti-phage and goat anti-mouse-HRP conjugate.

Phage-protein A was also checked for generating complexes with anti-insulin IgG. Phage-protein A (10¹²/ml) were combined with different concentrations of anti-insulin antibody and gently shaken at 37° C. for 30 minutes. The complexes were PEG precipitated before being applied to the ELISA plates that were coated with 0.25 mg/ml insulin overnight at 4° C. The bound phages were detected with either mouse anti-phage and goat anti-mouse antibodies, or with goat anti-mouse only. This second detection method was used to demonstrate that the anti-insulin antibody was indeed present in the complex together with phage protein A.

### In Vivo Studies

Nine month old PDAPP mice were given the phage-protA complexes (with Ab 196 or with anti-insulin Ab plus insulin) every 2 weeks (4 treatments) and then every month for the next 6 treatments, for a total of 10 treatments. The complexes were given by intranasal application, 20 microliters divided by the two nostrils. At the end of the treatments, the mice were euthanized and sacrificed. The left brain hemisphere from each mouse was frozen in liquid nitrogen. Each hemisphere was homogenized and divided into soluble and insoluble fractions. The insoluble fractions were later solubilized with guanidine-HCl. Both fractions were kept frozen until analyzed. Beta amyloid 1-42 was carried out by sandwich ELISA, using capture antibody and detection antibody, specific for Aβ 1-42. Determination of cytokines levels was carried out with specific kits for either IL1 beta or IL10 ((R&D systems).

### Results and Discussion

The protein A variant was synthesized using the primers depicted in FIG. 1A. The PCR product was sequenced to verify its integrity, and then cloned in pCANTAB5E. Phages that express the protein A variant fused to PIII of the phage were produced and used in ELISA.

Phages that Display Protein a Variant Bind Mainly IgG1 Antibodies

The protA phages were checked by ELISA for their binding to IgG type antibodies.

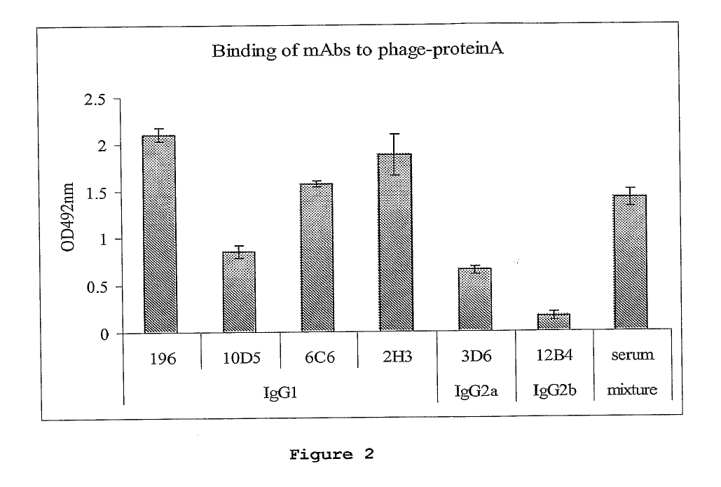

In FIG. 2, the binding of 10¹¹ phage particles to 15 nmol of different antibodies is presented.

Phage-protA-Bound Antibodies do not Disconnect from the Phage After they Bind their Antigens.

The antibodies were checked for whether or not they dissociate from the phage following binding to their antigen. As antigen, a biotinylated peptide of amino acid residues 1-16 from the N terminus of the β-amyloid peptide was used. The results of this experiment are summarized in FIG. 2. For this experiment, 10¹¹ phages displaying protein A were mixed with 10 μg (15 nmols) antibody in 0.1 M sodium phosphate pH 8.5. The mixture was gently shaken at 37° C. for 30 minutes and then the antigen (0.32 nmols of biotinylated Aβ 1-16) at a molar ratio of 50:1 in favor of the antibody was added to the mixture of phage-protein A and antibodies for an additional 30 minutes. The complexes were added to ELISA wells previously coated with rabbit anti-phage antibodies and blocked with 3% milk. Conjugated Avidin-HRP was used to detect complexes that bound the ELISA plate. Only phage-antibody complexes that were still connected after binding of the antigen would react with the Avidin-HRP conjugate. The serum used in these experiments was drawn from a mouse with titer against ERFH epitope.

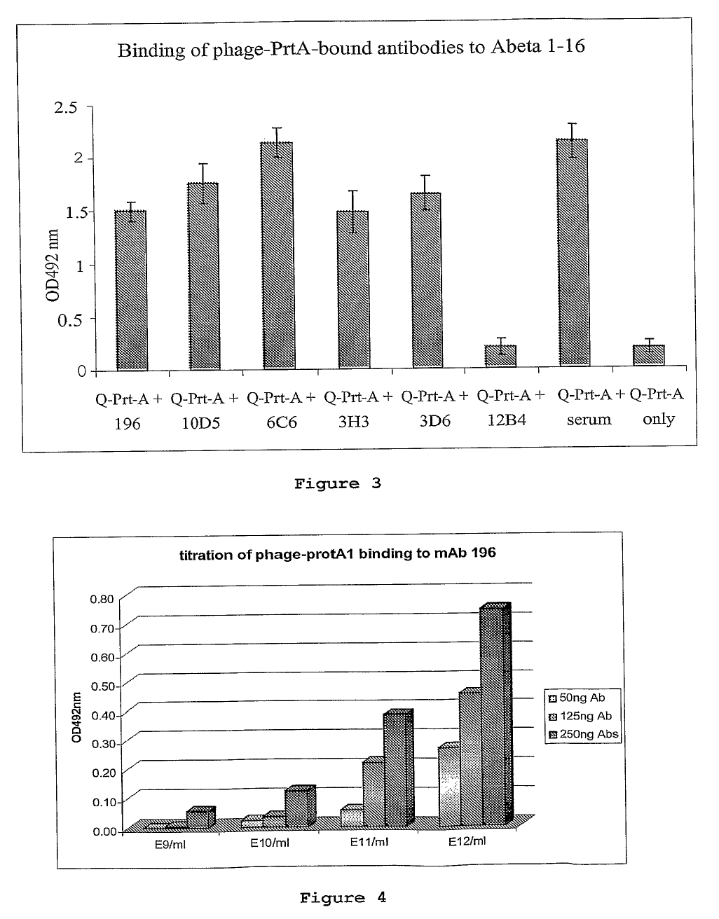

FIG. 3 demonstrates that when the protein A-bound antibodies bind an antigen they recognize, they do not detach from protein A. Antibody-antigen complexes that detach from the plate-bound phage—ProtA will be washed away and will not react with Avidin-HRP. As was shown in FIG. 2, 12B4 bound phage-ProtA weakly. It did not react here because it does not bind peptide 1-16 and was used as another negative control. Binding results did not change when the ELISA plate was washed with PBS buffer at pH 6.0. The lower pH did not cause dissociation of the antibody-antigen complex from phage-ProtA.

### Titration of the Binding of Protein A to mAb 196

The number of phages needed to bind different amounts of antibodies was titrated in order to verify that the binding is specific. In this experiment, antibody 196, which demonstrated the best binding capacity to protein A, was used to coat ELISA plates in 3 different concentrations, followed by blocking the wells with 3% milk. Different concentrations of phages-protein A were added and the bound phages were detected by rabbit anti-phage antibodies (FIG. 4). From FIG. 4, it is evident that the binding of phage-protein A to antibody 196 is specific; it is enhanced as a function of phage number and antibody concentration. In this experimental setting, saturated binding was not observed. In order to estimate the number of phages that display protein A, ELISA wells were coated with elevated phage concentrations and reacted with anti-phage antibodies (not shown). By comparing the OD at 492 between this analysis and the previous one presented in FIG. 4, it could be determined that approximately 30%-36% of the phages display protein A.

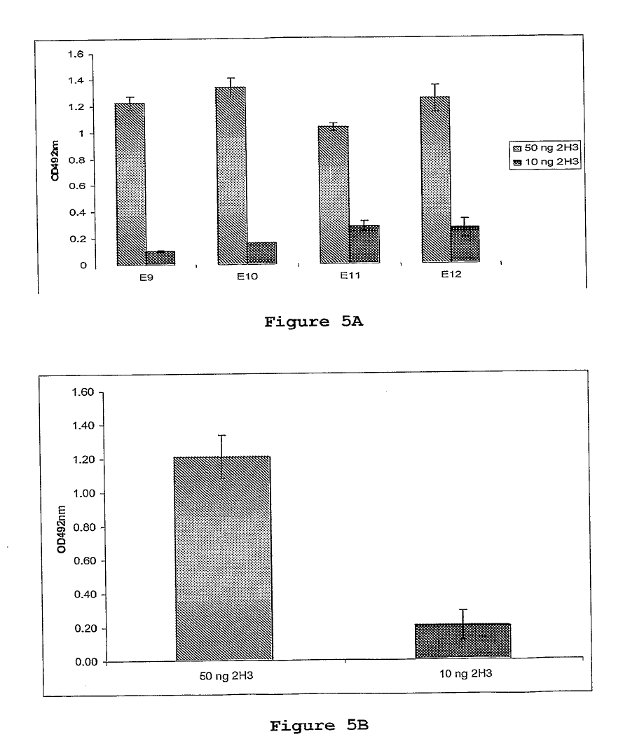

In order to determine the amount of antibody needed to bind a certain amount of phages (which will also indicate the amount of antigen that will be bound), another experiment was carried out in which elevated numbers of phages were combined with 2 separate concentrations of antibody (2H3), and 1:2 molar ratios of antibody:antigen (2 antigen molecules for each antibody molecule) (FIG. 5A). The mixtures were added to ELISA wells, previously coated with rabbit anti-phage antibodies, and coated with 3% milk in PBS. Detection of the bound phages was done with Avidin-HRP conjugate, which can only bind the biotinylated antigens.

Because the values observed for binding to each antibody concentration did not differ much between the different phage concentrations, the results can also be summarized as “average of averages” and plotted as shown in FIG. 5B.

These results demonstrate that the antibody concentration is the limiting factor in this experimental setting and not the phage concentration: at 10⁹/ml phage-protein A (5×10⁷ phages/50 μl used per ELISA well) there are enough protein A molecules to bind 50 ng which is approximately 70 μmol and they are not enough to saturate 5×10⁷ phage-protein A molecules.

### PEG-NaCl Precipitation Did not Cause Massive Phage-Antibody Dissociation

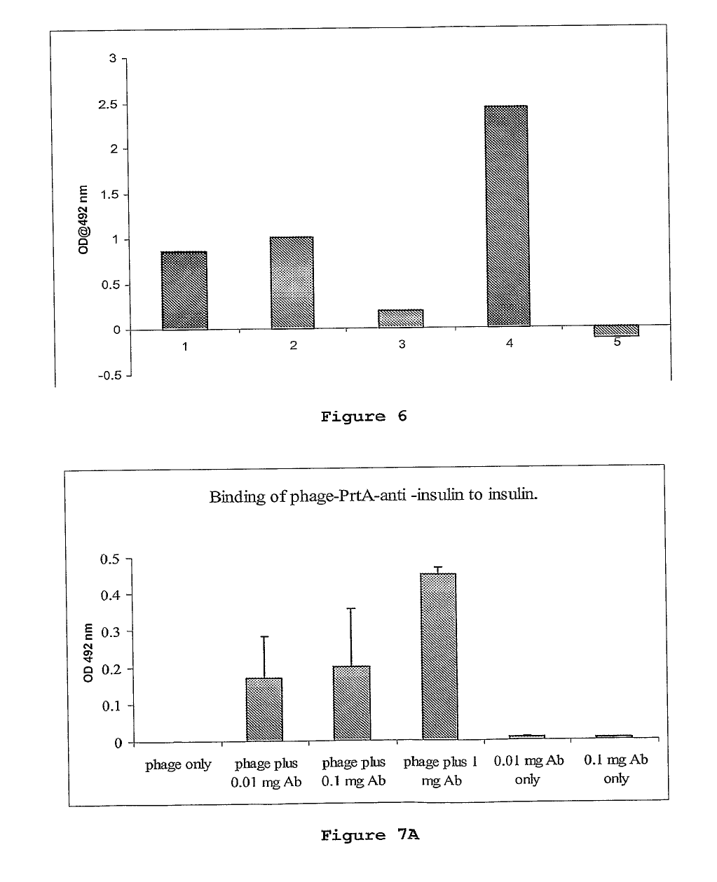

Whether or not PEG precipitation of phage-protein A-antibody complexes cause their dissociation was also checked. ELISA wells were coated with avidin and 1 μg/ml biotinylated 1-16 peptide. The wells were blocked with 3% milk in PBS. 10¹² phages were combined with 1 μg or with 5 μg antibody 196 in 0.1 M Na₂HPO₄ and gently shaken for 1 hr at 37° C. Half the mixture was precipitated using PEG-NaCl, followed by resuspension in the same buffer and same volume as before, and the other half was used without precipitation. Both mixtures were applied to the previously described ELISA plate (50 μl mixture/well) for 1 hr at 37° C. Detection of the bound phages was carried out with mouse anti-phage and goat anti-mouse-HRP conjugate. FIG. 6 shows that PEG precipitations caused about 20% of the phage protein A-antibody complexes to disassociate.

### Phage Protein A Binds Immunocomplex of Anti-Insulin Antibody and Insulin.

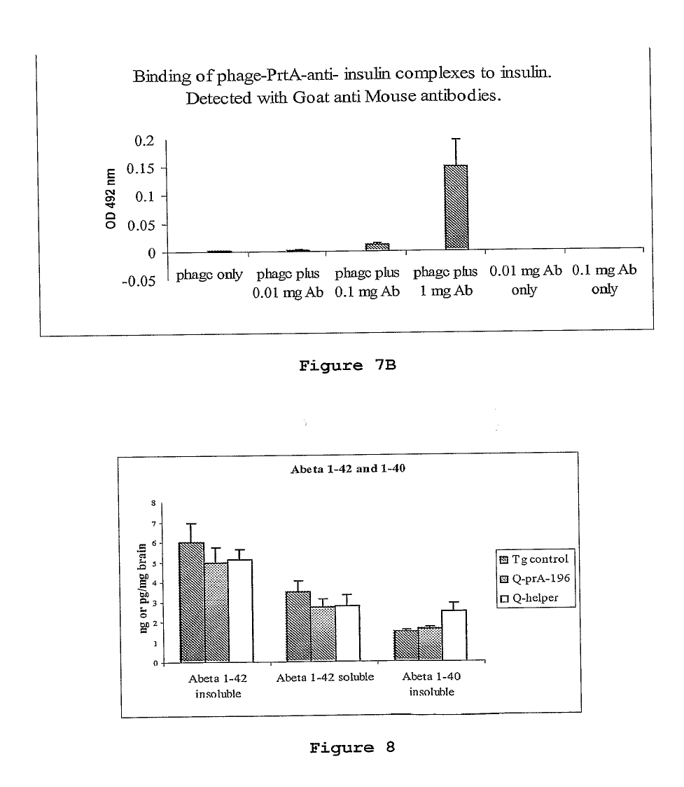

Phage-protein A was combined with anti-insulin IgG, and the complexes were used on ELISA plates that were coated with 0.25 mg/ml insulin. The complexes were PEG precipitated before being applied to the ELISA plate. The bound phages were detected with either mouse anti-phage and goat anti-mouse antibodies (FIG. 7A) or with goat anti-mouse only (FIG. 7B). This second detection method was used to demonstrate that the anti-insulin antibody was indeed present in the complex with phage protein A.

Phage-protein A (10¹²/ml) was combined with anti-insulin antibodies at different concentrations and gently shaken at 37° C. for 30 minutes. The complexes were PEG precipitated before being applied to the ELISA plates. The bound phages were detected with mouse anti-phage followed by goat anti-mouse antibodies.

The results of a similar assay as shown in FIG. 7A, but detected with goat anti-mouse antibodies only, is shown in FIG. 7B.

In summary, a filamentous phage that displays a variant of protein A that can bind antibodies of the IgG type was generated. As opposed to the native protein A of Staphylococcus aureus which strongly binds mouse IgG2a and IgG2b, this recombinant protein A binds mouse IgG1 molecules, which are the most abundant type, better. These phages can be used in vitro for purifying antibodies or, for example, can be used in vivo to deliver certain antibodies to the brain.

### In Vivo Studies

Phage-protA-196. Phage-protA-196 complexes were applied to PDAPP transgenic mice, as explained in the Materials and Methods section. The levels of β-amyloid peptide 1-42 in the soluble and the insoluble fractions (aggregated peptides and membranes that were solubilized with guanidine-HCl) and the levels of Aβ 1-40 in the insoluble fractions were checked. An approximate 20% reduction in the amount of Aβ 1-42 in both soluble and insoluble fractions were observed in treated mice compared to transgenic non-treated controls. However, the results were not significant (FIG. 8). In Aβ 1-40, no difference was observed between mice treated with the phage complexes compared to control untreated mice.

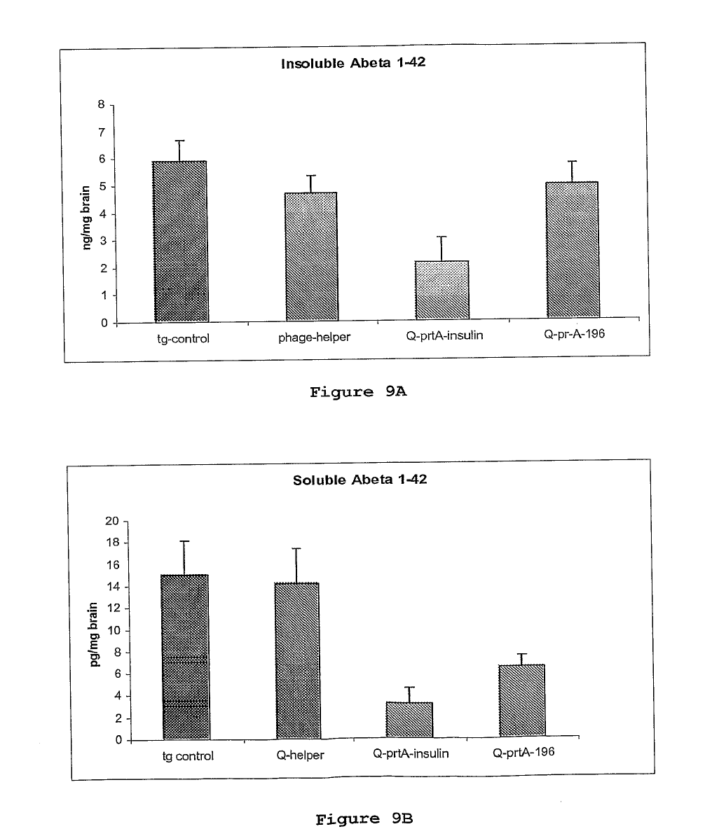

Phage-protA-anti-insulin-insulin PDAPP mice were treated with phage-antibody-insulin complexes. In FIG. 9A, a more dramatic difference in the level of insoluble Aβ 1-42, a reduction of about 63% in mice treated with complex compared to Tg non-treated mice or to mice treated with phage-196 complexes, was observed. Mice treated with phage only had an average 23% reduction in Aβ 1-42 levels. The reduction in the level of soluble Aβ 1-42 in treated mice was even more dramatic and reached 80% compared to controls (FIG. 9B).

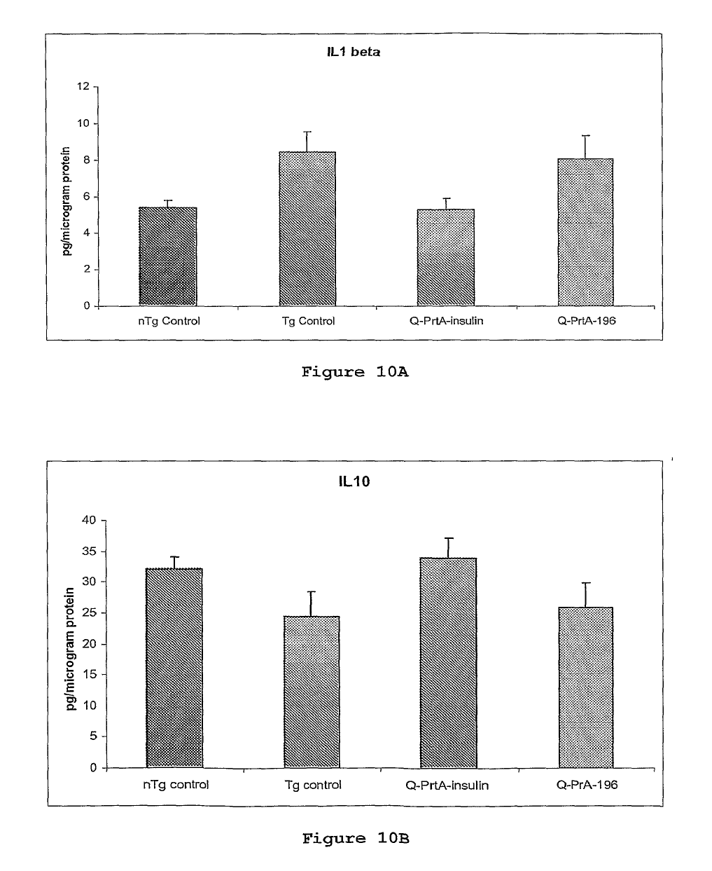

The levels of IL1β, a pro-inflammatory cytokine, and IL10, an anti-inflammatory cytokine, were checked. The levels of IL1β in transgenic non-treated mice were about 36% higher than the levels of IL1β in non-transgenic mice (FIG. 10A), meaning that the disease caused inflammation that is manifested in higher IL1β concentrations. The complex-treated mice, however, contained a level similar to the levels of IL1β in non-transgenic mice, suggesting that the treated mice did not develop brain inflammation. The same phenomenon was observed in the concentrations of the anti-inflammatory cytokine, IL10 (FIG. 10B). Here, again, the levels of IL10 were similar both in the non-transgenic mice and in the transgenic treated mice, compared to transgenic control mice which contained on average 18% less IL10. Cytokine levels in PDAPP mice that received phage-protA-196 showed no difference between treated mice and transgenic controls (data not shown).

Previous studies have suggested an acutely improving effect of insulin on memory function via the intranasal route of insulin administration known to provide direct access of the substance to the cerebrospinal fluid compartment (Strachan, 2005). Previous results indicate a prevalence of insulin receptors in limbic and hippocampal regions, as well as improvements in memory with systemic insulin. Insulin was combined with phages to demonstrate that an increased number of phages are introduced to the brain, leading to effective reduction in amyloid burden. The effect is accompanied by improvement in cytokines profile in the brain. This cumulative effect may be an efficient way to take anti-aggregating properties of the phages plus better penetration via insulin as a protective treatment of AD.

Having now fully described this invention, it will be appreciated by those skilled in the art that the same can be performed within a wide range of equivalent parameters, concentrations, and conditions without departing from the spirit and scope of the invention and without undue experimentation.

While this invention has been described in connection with specific embodiments thereof, it will be understood that it is capable of further modifications. This application is intended to cover any variations, uses, or adaptations of the inventions following, in general, the principles of the invention and including such departures from the present disclosure as come within known or customary practice within the art to which the invention pertains and as may be applied to the essential features hereinbefore set forth as follows in the scope of the appended claims.

All references cited herein, including journal articles or abstracts, published or corresponding U.S. or foreign patent applications, issued U.S. or foreign patents, or any other references, are entirely incorporated by reference herein, including all data, tables, figures, and text presented in the cited references. Additionally, the entire contents of the references cited within the references cited herein are also entirely incorporated by references.

Reference to known method steps, conventional methods steps, known methods or conventional methods is not in any way an admission that any aspect, description or embodiment of the present invention is disclosed, taught or suggested in the relevant art.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

### REFERFENCES

Adzamil et al., Inves. Radiol. 26 (2):143-8, 1991
Benhar et al., Phage display of single-chain antibodies. In: J. Colligen (Ed), Current Proteocols in Immunology, Vol. 10.19B, John Wiley & Sons, Inc, USA (2001)
Berdichevsky et al., J. Immunol. Methods, 228:151-62 (1999)
Bluthner et al, “Mapping of epitopes recognized by PM/Sc1 autoantibodies with gene-fragment phage display libraries”, J Immunol Methods 198:187-198 (1996)
Bonnycastle et al., “Probing the basis of antibody reactivity with a panel of constrained peptide libraries displayed by filamentous phage. J Mol. Biol. 24; 258(5):747-62 (1996)
Brent et al., Current Protocols in Molecular Biology, John Wiley & Sons Inc. (2003)
Caughey et al, “Secondary structure analysis of the scrapie-associated protein PrP 27-30 in water by infrared spectroscopy”, Biochemistry 30:7672-7680 (1991)
Chartier Harlan et al, Nature 353:844 (1991)
Chou et al, Biopharm Drug Dispos. 18(4):335-46 (1997)
Cortese et al, “Epitope discovery using peptide libraries displayed on phage”, Trends Biotechnol 12:262-267 (1994)
Cortese et al, “Identification of biologically active peptides using random libraries displayed on phage”, Curr Opin Biotechnol 6:73-80 (1995)
Cortese et al, “Selection of biologically active peptides by phage display of random peptide libraries. Curr Opin Biotechnol 7:616-621 (1996)
Cwirla et al, “Peptides on phage: a vast library of peptides for identifying ligands”, Proc Natl Acad sci USA 87:6378-6382 (1990)
De Gioia et al, “Conformational polymorphism of the amyloidogenic and neurotoxic peptide homologous to residues 106-126 of the prion protein”, J Biol Chem 269:7859-7862 (1994)
Djojonegoro B M, Benedik M J, Willson R C., “Bacteriophage surface display of an immunoglobulin-binding domain of Staphylococcus aureus protein A” Biotechnology 12(2):169-72 (1994).
Dotto et al, “The functional origin of bacteriophage f1 DNA replication: Its Signal and domains”, J Mol Biol 172:507-521 (1984)
Dower W J, “Phage power”, Curr Biol 2:251-253 (1992)
Draghia et al, Gene Therapy 2:418-423 (1995)
Felici et al, “Selection of antibody ligands from a large library of oligopeptides expressed on a multivalent exposition vector”, J Mol Biol 222:301-310 (1991)
Frenkel et al, “N-terminal EFRH sequence of Alzheimer's β-amyloid peptide represents the epitope of its anti-aggregating antibodies”, J Neuroimmunology 88:85-90 (1998)
Frenkel, D., Solomon, B. and Benhar, I. “Modulation of Alzheimer's beta-amyloid neurotoxicity by site-directed single-chain antibody” J Neuroimmunol, 106:23-31 (2000a)
Frenkel, D., Katz, O. and Solomon, B. “Immunization against Alzheimer's β-amyloid plaques via EFRH phage administration” PNAS 97(21):11455-11459 (2000b).
Frenkel, D., Kariv, N. and Solomon, B. “Generation of auto-antibodies towards Alzheimer's disease vaccination” Vaccine 19, 2615-2619 (2001).
Frenkel and Solomon, PNAS, 99:5675-5679 (2002)
Gajdusek, Science 197:943-960 (1991)
Gazit E., “Mechanistic studies of the process of amyloid fibrils formation by the use of peptide fragments and analogues: implications for the design of fibrillization inhibitors” Curr Med. Chem. 9(19):1725-35 (2002)
Goding J W., “Use of staphylococcal protein A as an immunological reagent” J Immunol Methods. 20:241-53 (1978)
Goldsmith et al, “Adsorption protein of bacteriophage fd: Isolation, molecular properties, and location in virus”, Biochemistry 16:2686-2694 (1977)
Goate et al, Nature 349:704 (1991)
Goudswaard J, van der Donk J A, Noordzij A, van Dam R H, Vaerman J P. “Protein A reactivity of various mammalian immunoglobulins” J Scan Immunol. 8(1):21-8 (1978)
Graille M, Stura E A, Corper A L, Sutton B J, Taussig M J, Charbonnier J B, Silverman G J. “Crystal structure of a Staphylococcus aureus protein A domain complexed with the Fab fragment of a human IgM antibody: structural basis for recognition of B-cell receptors and superantigen activity” Proc Natl Acad Sci USA. 97(10):5399-404 (2000)
Gray et al “Adsorption complex of filamentous fd virus”, J Mol Biol 146:621-627 (1981)
Greenwood et al, “Multiple display of foreign peptides on a filamentous bacteriophage”, J Mol Biol 220:821-827 (1991)
Hardy, TINS, 20:154 (1997)
Hoess et al, “Identification of a peptide which binds to the carbohydrate-specific monoclonal antibody B3”, Gene 128:43-49 (1993)
Holliger et al, “A conserved infection pathway for filamentous bacteriophages is suggested by the structure of the membrane penetration domain of the minor coat protein g3p from phage fd”, Structure 5:265-275 (1997)
Hoogenboom, H. R., de Bruine, A. P., Hufton, S. E., Hoet, R. M. Arends, J. W. and Roovers, R. C. “Antibody phage display technology and its applications” Immunotechnology 4:1-20 (1998).
Horiuchi and Caughey, “Specific binding of normal prion protein to the scrapie form via a localized domain initiates its conversion to the protease-resistant state”, EMBO J. 18:3193-3203 (1999)
Iannolo et al., “Modifying filamentous phage capsid: limits in the size of the major capsid protein” J. Mol. Biol., May 12; 248(4):835-44 (1995)
Kay et al, “An M13 phage library displaying 38-amino-acid peptides as a source of novel sequences with affinity to selected targets”, Gene 128:59-65 (1993)
Kay et al., Phage Display of Peptides and Proteins, A Laboratory Manual, Academic Press (1996)
Kanyo et al, “Antibody binding defines a structure for an epitope that participates in the PrPC-->PrPSc conformational change”, J Mol. Biol. 293:855-863 (1999)
Kim et al, “Viable deletions of the M13 complementary strand origin”, Proc Natl Acad Sci USA 78:6784-6788 (1981)
Koivunen et al, “Phage libraries displaying cyclic peptides with different ring sizes: ligand specificities of the RGD-directed integrins”, Biotechnology 13:265-270 (1995)
Krebber et al, “Co-selection of cognate-antigen pairs by selectively-infective phages”, FEBS Lett 377:227-231 (1995)
Li Y, Cockburn W, Whitelam G C, Filamentous bacteriophage display of a bifunctional protein A::scFv fusion. Mol. Biotechnol. 1998 June; 9(3):187-93.
Luzzago et al, “Mimicking of discontinuous epitopes by phage displayed peptides, α. Epitope mapping of human H ferritin using a phage library of constrained peptides”, Gene 128:51-57 (1993)
Maggio J E, Mantyh P W, “Brain amyloid—a physicochemical perspective” Brain Pathol. 6(2):147-62 (1996)
Marvin et al, “Molecular model and structural comparisons of native and mutant class filamentous bacteriophages Ff (fd, f1, M13), f1 and Ke”, J Mol Biol 235:260-286 (1994)
Mathison et al, J. Drug Target, 5(6):415-441 (1998)
Matthews et al, “Substrate phage: selection of protease substrates by monovalent phage display”, Science, 260:1113-1116 (1993)
McCafferty et al, “Phage enzymes: expression and affinity chromatography of functional alkaline phosphatase on the surface of bacteriophage”, Protein Eng 4:955-961 (1992)
McCafferty et al., “Phage antibodies: filamentous phage displaying antibody variable domains” Nature, 348(6301):552-4 (1990)
Medynski, D. “Phage display: all dressed up and ready to role” Biol Technol 12, 1134-1136 (1994).
Meola, A., Delmastro, P., Monaci, P et al. “Derivation of vaccines from mimotopes: Immunological properties of human B virus surface antigen mimotopes displayed on filamentous phage” J Immuno 154:3162-3172 (1995).
Messing J, “New M13 vectors for cloning”, Methods Enzymol 101:20-78 (1983)
Model et al, “Filamentous Bacteriophage”, in The Bacteriophages, Calendar R (ed.), Plenum Press, New York and London, Vol. 2, p. 375 (1988)
Monaci P., et al., Curr Opin Mol. Ther., 3(2):159-69 (2001)
Moses et al, “Restructuring the bacteriophage f1 genome: Expression of gene VIII in the intergenic space”, Virology 104:267-278 (1980)
Mullan et al, Nature Genet. 1:345 (1992)
Murrell et al, Science 254:97 (1991)
Pan et al, “Conversion of alpha-helices into beta-sheets features in the formation of the scrapie prion proteins”, Proc Natl Acad Sci USA 90:10962-10966 (1993)
Parmley et al, “Antibody-selectable filamentous fd phage vectors: affinity purification of target genes”, Gene 73:305-318 (1988)
Peretz et al, “A conformational transition at the N terminus of the prion protein features in formation of the scrapie isoform”, J Mol Biol 273:614-622 (1997)
Rasqualini et al, “Organ targeting in vivo using phage display peptide libraries”, Nature 380:364-366 (1996)
Saggio et al., Gene, 152:35-39 (1995)
Sambrook et al., Molecular cloning: A laboratory manual, Cold Spring Harbor Laboratory, Cold Spring Harbor, N.Y. (1989)
Sampath A, Abrol S, Chaudhary V K., “Versatile vectors for direct cloning and ligation-independent cloning of PCR-amplified fragments for surface display on filamentous bacteriophages” Gene. 190(1):5-10 (1997)
Schmitz et al, “Catalytic specificity of phosphotyrosine kinases Blk, Lyn, c-Src and Syk as assessed by phage display”, J Mol Biol 260:664-677 (1996)
Scott et al, “Searching for peptide ligands with an epitope library”, Science 249:386-390 (1990)
Scott, J. K. “Discovering peptide ligands using epitope libraries” Trends in Biochem Sci, 241-245 (1992)
Selvaggini et al, “Molecular characteristics of a protease-resistant, amyloidogenic and neurotoxic peptide homologous to residues 106-126 of the prion protein”, Biochem Biophys Res Commun 194:1380-1386 (1993)
Silen and Agard, “The alpha-lytic protease pro-region does not require a physical linkage to activate the protease domain in vivo”, Nature 341:462-464 (1989)
Smith G P “Surface display and peptide libraries”, Gene 128:1-2 (1993)
Smith D H. et al. “Accumulation of Amyloid and Tau and the Formation of Neurofilament Inclusions Following Diffuse Brain Injury in the Pig” J. Neuropathol. Exp. Neurol., 58(9):982-992 (1999)
Sparks et al, “Identification and characterization of Src SH3 ligands from phage-displayed random peptide libraries”, J Biol Chem 269:23853-23856 (1994)
Strachan M. W. J., “Insulin and cognitive function in humans: experimental data and therapeutic considerations” Biochemical Society Transactions. 33:1037-1040 (2005)
Sugimoto et al, “Studies on bacteriophage fd DNA. IV. The sequence of messenger RNA for the major coat protein gene”, J Mol Biol 111:487-507 (1977)
Tagliavini et al, “Synthetic peptides homologous to prion protein residues 106-147 form amyloid-like fibrils in vitro”, Proc Natl Acad Sci USA 90:9678-9682 (1993)
Uppala and Koivunen, Chem. High Throughput Screen, 3:373-392 (2000)
Van Wezenbeek et al, “Nucleotide sequence of the filamentous bacteriophage M13 DNA genome: comparison with phage fd”, Gene 11:129-148 (1980)
Wilesmith and Wells, Curr Top Microbiol Immunol 172:21-38 (1991)
Willis et al., “Immunological properties of foreign peptides in multiple display on a filamentous bacteriophage”, Gene (1993)
Winter, G., Griffiths, A. D., Hawkins, R. E. and Hoogenboom, H. R. “Making antibody by phage display technology” Annu Rev Immunol 12:433-455 (1994).
Wrighton et al, “Small peptides as potent mimetics of the protein hormone erythropoietin”, Science 273:458-463 (1996)
Zacher et al, “A new filamentous phage cloning vector: fd-tet”, Gene 9:127-140 (1980)

1. A phage display vehicle, comprising a filamentous bacteriophage displaying on its surface as a non-native filamentous bacteriophage molecule, protein A, or a fragment or variant thereof capable of binding the Fc portion of antibodies, and an antibody or an antigen-antibody immunocomplex bound to said protein A or fragment or variant thereof by its Fc portion, with the proviso that the filamentous bacteriophage is not also conjugated through a linker or directly to a drug and that the antibody or immunocomplex is not also immobilized on a solid carrier.

2. The phage display vehicle of claim 1, wherein the filamentous bacteriophage is selected from the group consisting of M13, f1 and fd bacteriophage, and any mixture thereof.

3. The phage display vehicle of claim 1, wherein the filamentous bacteriophage is M13.

4. The phage display vehicle of claim 1, wherein the antibody is of the IgG class.

5. The phage display vehicle of claim 1, wherein the antibody bound to said protein A or fragment or variant thereof is a monoclonal antibody.

6. The phage display vehicle of claim 5, wherein said monoclonal antibody is a monoclonal antibody raised against or specific for the amyloid β peptide of Aβ1-42 or β1-40.

7. The phage display vehicle of claim 5, wherein said monoclonal antibody is specific for a target molecule associated with a brain disease, disorder or condition.

8. The phage display vehicle of claim 7, wherein said brain disease, disorder or condition is a plaque-forming disease or disorder.

9. The phage display vehicle of claim 7, wherein said monoclonal antibody is labeled.

10. The phage display vehicle of claim 1, wherein said immunocomplex bound to said protein A or a fragment or variant thereof is an immunocomplex of insulin and an anti-insulin antibody.

11. The phage display vehicle of claim 1, wherein said non-native filamentous bacteriophage molecule displayed on the surface of said filamentous bacteriophage consists of protein A, or a fragment or variant thereof capable of binding the Fc portion of antibodies, bound to an antibody or antigen-antibody immunocomplex.

12. A phage display vehicle, comprising a filamentous bacteriophage displaying on its surface as a non-native filamentous bacteriophage molecule, protein A, or a fragment or variant thereof capable of binding the Fc portion of antibodies, and an antibody bound to said protein A or fragment or variant thereof by its Fc portion, wherein said antibody is specific for a molecule that is not presented as a target on a cell surface and with the proviso that the antibody is not also immobilized on a solid carrier.

13. The phage display vehicle of claim 12, wherein the filamentous bacteriophage is selected from the group consisting of M13, f1 and fd bacteriophage, and any mixture thereof.

14. The phage display vehicle of claim 12, wherein the filamentous bacteriophage is M13.

15. The phage display vehicle of claim 12, wherein the antibody is of the IgG class.

16. The phage display vehicle of claim 12, wherein the antibody bound to said protein A or fragment or variant thereof is a monoclonal antibody.

17. The phage display vehicle of claim 16, wherein said monoclonal antibody is a monoclonal antibody raised against or specific for the amyloid β peptide of Aβ1-42 or β1-40.

18. The phage display vehicle of claim 16, wherein said monoclonal antibody is specific for a target molecule associated with a brain disease, disorder or condition.

19. The phage display vehicle of claim 18, wherein said brain disease, disorder or condition is a plaque-forming disease or disorder.

20. A pharmaceutical composition, comprising the phage display vehicle of claim 1 and a pharmaceutically acceptable carrier, excipient, diluent or auxiliary agent.

21. A method for treating or inhibiting a brain disease, disorder or condition, comprising intranasally administering an effective amount of the phage display vehicle of claim 1 to a subject in need thereof to inhibit or treat the brain disease, disorder or condition.

22. The method of claim 21, wherein said brain disease, disorder or condition is a plaque-forming disease or disorder.

23. The method of claim 22, wherein the plaque-forming disease or disorder is Alzheimer's disease.

24. The method of claim 23, wherein the antibody bound to said protein A or fragment or variant thereof displayed on the phage display vehicle is an antibody specific for an amyloid β peptide.

25. The method of claim 24, wherein said antibody is specific for amyloid β peptide 1-42 or 1-40.

26. The method of claim 22, wherein the plaque-forming disease or disorder is a prion disease.

27. The method of claim 21, wherein said brain disease, disorder or condition is a brain tumor.

28. The method of claim 21, wherein said brain disease, disorder or condition is a brain inflammatory disease, disorder or condition.

29. The method of claim 21, wherein the subject is a mammal.

30. The method of claim 21, wherein the subject is a human.

31. The method of claim 21, wherein said immunocomplex bound to said protein A or fragment or variant thereof is an immunocomplex of insulin and an anti-insulin antibody.

32. A method for diagnosing a brain disease, disorder or condition, comprising: intranasally administering the phage display vehicle of claim 1 to a subject in need thereof, and detecting the phage display vehicle with its antibody component bound to a target molecule associated with a brain disease, disorder or condition to diagnose the presence of the brain disease, disorder or condition.

33. The method of claim 32, wherein the antibody is detectably labeled.

34. The method of claim 33, wherein the antibody is detectably labeled with a radionuclide.

35. The method of claim 33, wherein the antibody is detectably labeled with a contrast agent.

36. The phage display vehicle of claim 1, wherein said antibody is specific for IL-6.

37. The pharmaceutical composition of claim 20, wherein said antibody is specific for IL-6.

38. The method of claim 28, wherein said antibody is specific for IL-6 and the brain inflammation is the result of stroke, brain injury or other neurodegenerative disease.
